(19) United States
(12) Patent Application Publication
(10) Pub. No.: US 2010/0016668 A1
(43) Pub. Date: Jan. 21, 2010

(54) MEDICAL DEVICE FOR DISCREETLY PERFORMING A ROUTINE VAGINAL EXAMINATION

(75) Inventor: Ehud Gal, Reut (IL)

Correspondence Address: MARSTELLER & ASSOCIATES, P O BOX 803302, DALLAS, TX, 75380-3302, US

(73) Assignees: WAVE GROUP LTD., Tel Aviv (IL); O.D.F. MEDICAL LTD., Tel Aviv (IL)

(21) Appl. No.: 12/374,791

(22) PCT Filed: Jul. 23, 2007
(86) PCT No.: PCT/IL2007/000927

§ 371 Date: Mar. 8, 2009

(30) Foreign Application Priority Data
Jul. 24, 2006 (IL) 177040

(51) Int. Cl. A61B 1/307

(52) U.S. Cl. 600/135

(57) ABSTRACT

The invention is a system for performing a routine vaginal examination. The examination is carried out using a medical imaging device that is optimally shaped to match the shape of the vaginal canal and comprises an imaging sensor, wide field of view optics, illumination means, electronic circuitry, and communication means. In addition a medical image and analysis (MIUA) unit comprising a processor and a dedicated MIUA algorithm is provided, either as part of the device or as a separate unit. The system is characterized in that the dedicated MIUA algorithm compares the acquired images to a predefined standard of quality and verifies that sufficient images of a predefined region of interest (ROI) have been obtained; saves the acquired images that meet the predefined standard and also show the ROI; and signals the user carrying out the examination when a sufficient number of images having sufficient image quality and taken at appropriate viewing angles of the area being examined have been acquired. The device and MIUA unit enable routine examination of the vagina to be carried out at any location by untrained persons, preferably by the woman herself in the privacy of her own home. The images acquired by the camera are transmitted to an authorized center where trained medical personnel view, analyze, and interpret them.

### FIELD OF THE INVENTION

The present invention is related to the field of medical devices. More specifically, the invention is related to the field of personal medical devices used to discreetly conduct a routine vaginal examination in a simple manner.

### BACKGROUND OF THE INVENTION

Nowadays, periodical vaginal examinations take place routinely for women of almost all ages in the western world. Depending on the age of the woman, physicians normally recommend conducting vaginal examinations once or twice a year. The examination is usually performed for early detection of cervical cancer as well as other abnormalities. Three main areas are usually examined by the physician; the cervix, the vaginal canal and the vulva. The examinations are usually conducted on a gynecologist's chair in which the woman's legs are spread in the lithotomic position and the gynecologist inspects the vaginal area, usually using a mechanical device called a “speculum”. These examinations are considered to be intrusive, inconvenient, and embarrassing by most women.

Another type of vaginal examination is the prenatal examination. During woman's labor pains, a prenatal examination is usually performed in order to determine whether the time has come to deliver the baby. The examiner in this procedure is usually a physician or a midwife who feels the opening diameter of the cervix with their fingers, in addition to measuring other parameters, to decide whether the time to deliver has come. If it is still not time to deliver, the woman is usually sent back home until the next labor pains arrive. Sometimes, especially during a woman's first pregnancy, this process is repeated several times until the actual time to deliver comes. This process suffers from impreciseness and increases the chance of infections proportionally to the number of examinations.

Yet another kind of vaginal examination is made to detect conditions where there is suspicion of a premature delivery. Premature delivery is a relatively common phenomenon. Today advanced tests are conducted to detect signs for a possible premature delivery using “reagents”, i.e. an indicator which changes its color when coming in contact with a specific substance; a reagent sensitive to fibronectin assists in the detection of a chance of a premature delivery.

An advanced medical device capable of a wide Field Of View (Hereinafter: “FOV”) can improve the vaginal examinations made today. Optics which provides a wide field of view enables acquisition of video images for better orientation of the examined area and assists with early detection of abnormalities. This type of optics can enable a FOV which is wider than the standard (about 45 degrees horizontally and vertically) and up to an almost full 360 degree FOV (“Omni-Directional” FOV).

Special optical systems enabling wide FOV as well as Omni-directional FOV are abundant. There are many commercial optical systems enabling a wide FOV, some of which are called by those skilled in the art “Fish Eye” lenses. Lenses providing up to about a 180 degree FOV are known as half sphere or semispherical FOV lenses. In addition, some advanced optic lenses enable observation at a selected preplanned FOV; such optics enable a FOV of almost up to 360 degrees and some enable in addition zoom capabilities at predefined regions of interest within the selected FOV. The information required by the device can be displayed on a computer screen using special software and a Man Machine Interface in order to assist the observer to orient himself in the wide FOV images.

Typical examples of methods of applying Omni-directional imaging using special optics and methods are described in the following publications:
U.S. Pat. No. 6,304,285 To Geng—The invention described is a hemi spherical mirror and a projector with a changeable wavelength filter which is positioned in accordance to the said mirror thus enabling the acquiring of a 180 degree FOV.
U.S. Pat. No. 5,790,182 To St. Hilaire—Describes two mirrors positioned relative to one another at the “Golden Relation” thus providing a wide field of view.
PCT Publication WO 02/059676 To Gal—Refers to lenses with asymmetric convex surfaces enabling a wide FOV.
PCT Publication WO 03/026272 To Gal—Refers to lenses based on an asymmetric surface and a symmetric surface.
PCT Publication WO 04/042428 To Gal—Refers to lenses which enable acquiring an Omni-directional FOV and in addition to provide Omni-directional illumination of the lens's FOV simultaneously.
PCT Publication WO 04/008185 To Gal—Refers to Omni-Directional observation using a central asymmetric lens and additional lenses in accordance.

There are many additional methods to apply wide FOV optics to imaging sensors. For the sake of brevity and since it is not the essence of the present invention, no further discussion of the subject shall be described herein.

Many different video-based systems are described in the prior art for conducting vaginal examinations. Representative of these systems are:
U.S. Pat. No. 4,979,498 to Oneda, et al., which describes a video cervioscope system for examination of the cervix comprising an elongated tubular member having a light guide and imaging means at the distal end of said tubular member and a disposable sleeve.
PCT application WO 05/19950 to GAL describes a device which enables the health care provider to refine vaginal examinations. The device consists among other things of a disposable cover which enables isolation of the front assembly, which is inserted into the vaginal canal, from the surrounding tissue and the body fluids. The front assembly consists of an imaging device, optics, and illumination means which enable acquisition of images from within the inspected area. The cover also enables insertion of tools during the treatment and simplifies the sterilization process by replacing it after the treatment as a disposable component.
U.S. Pat. No. 6,896,653 to Vail III, et al. refers to a personal pelvic viewer which a woman may place into her own vagina to view and record images of the interior of her vagina and cervix on a remote monitor.

It is felt by the inventor that, while most women would prefer the option of replacing routine vaginal examinations that are currently carried out in clinics or doctor's offices with a more discreet self-examination, they are neither sufficiently trained nor interested in viewing the images or in analyzing the results of the examination. What is needed, and presently unavailable, is a method which combines the advantages of self-examination with that of professional analysis and interpretation of the results.

It is a purpose of the present invention to provide a method and device which supplies the above mentioned need.

It is another purpose of the present invention to provide a personal device for discreetly carrying out a routine vaginal examination.

It is another purpose of the present invention to provide a device enabling simple operation based on the use of Medical Image Understanding and Analysis software.

It is yet another purpose of the present invention to provide a device which reduces the workload of clinical resources by enabling routine vaginal examinations to be carried out by the woman herself and by using advanced methods to assist in gathering, documenting, and classifying the acquired data.

Further advantages of the invention shall come apparent as the description proceeds.

### SUMMARY OF THE INVENTION

In a first aspect the invention is a system for performing a routine vaginal examination. The system comprises:
a medical imaging device comprising: optics and an imaging sensor having a Wide Field of View (WFOV) capability; circuitry; illumination means; and communication means, the device designed for insertion into the vagina;
a medical image understanding and analysis (MIUA) unit comprising a processor, and a dedicated MIUA algorithm; and
an authorized center comprising image viewing software and human experts to analyze the images acquired by the device.

The system is characterized in that the dedicated MIUA algorithm of the MIUA unit compares the acquired images to a predefined standard of quality verifies that sufficient images of a predefined region of interest (ROI) has been obtained; saves the acquired images that meet said predefined standard and include all of the predefined ROI; and provides an indication to the user carrying out said examination when a complete set of “Eligible Views” of the examined area has been obtained.

The dedicated MIUA algorithm of the system of the invention is adapted such that the routine vaginal examination can be carried out by an inexperienced user. The inexperienced user can be the woman being examined, i.e. the system allows self-examination.

The medical imaging device comprises an elongated insertion section shaped so that it can be easily and smoothly inserted into the vagina attached to a handle shaped to assist the user to insert the insertion section into the vagina and to maneuver it in order to obtain “Eligible Views”. At least the distal part of the insertion section is optically transparent to the wavelengths produced by the illumination means and to which the imaging sensor is sensitive. The device may optionally comprise a disposable sterile cover, which slides over at least the part of the device that is inserted into the vagina or comes in contact with the body of the person being examined. At least the distal part of the disposable cover must be optically transparent to the wavelengths produced by the illumination means and to which the imaging sensor is sensitive. Optionally one or more sensors or indicators may be attached to the outer surface of the insertion section or the disposable cover of the device in order to detect abnormalities in the secretions inside the vagina.

The imaging sensor, the optics and the illumination means may be adapted to gather images at different wavelengths to enhance detection or recognition of pathologies. In an embodiment of the system, the imaging sensor is a black and white sensor and the illumination means provide illumination at different wave lengths to provide multi-spectral imaging to enhance detection or recognition of pathologies. The imaging device may comprise at least one filter to assist in detection of pathologies using the acquired information. In an embodiment of the imaging device the filter function is provided by designated software.

In embodiments of the system of the invention the MIUA unit comprises a display and interface with the user. In one embodiment the MIUA unit is based on a standard personal computer on which is installed the MIUA algorithm. In another embodiment the MIUA unit is an integral part of the medical imaging device.

In embodiments of the system the MIUA unit is adapted to provide the user with instructions how to maneuver the device in order to obtain a complete set of “Eligible Views” and/or to enable modification of the definition of the parameters of electrical components of the circuitry within the device in order to improve the image quality. Examples of parameters that can be modified are related to one or more of the following: illumination intensity, the exposure time of the imaging sensor, the focus area, optical zoom, or digital zoom.

After a complete set of “Eligible Views” has been collected, either the MIUA unit can automatically transmit data comprising at least the set of “Eligible Views” to the authorized center and signal the user that the examination has been completed or, in another embodiment, the MIUA unit signals the user that the examination has been completed and the user manually transmits data comprising at least the set to the authorized center. In either case, the data transmission, both between imaging device and the MIUA unit and the MIUA unit and the authorized center can be performed over a standard communication network. Preferably the device includes a unique ID address transmitted with the data to the authorized center.

The MIUA unit can be adapted to provide images of the entire interior of the vagina and/or to recognize and provide images of specific areas in the vagina, e.g. the cervix. In an embodiment the MIUA unit enables measurement of the opening of the cervix before childbirth.

In embodiments of the system the human expert at the authorized center is the physician of the woman who was examined. In embodiments of the system the authorized center comprises one or more computers containing software adapted to provide different formats for observation of the data received, for documentation of the data, for comparison of the data to the results of previous examinations of the same woman, and for consolidation of data for wider statistical studies by human experts.

In another aspect the invention is a method for using the system of the first aspect for performing a routine vaginal examination. The method comprises:
the user turning on the medical imaging device;
the user inserting the insertion section of the medical imaging device into the vagina to be examined;
the user withdrawing the insertion section from the vagina upon receiving a signal from the MIUA unit that a set of “Eligible Views” has been obtained;
transmitting data comprising at least the set of “Eligible Views” to the authorized center;
the user turning off the medical imaging device; and
analyzing the transmitted data at the authorized center.

In embodiments of the invention, the user follows instructions from the MIUA unit to maneuver the insertion section to change the orientation of the imaging sensor within the vagina.

In preferred embodiments of the method of the invention the user is the woman being examined.

In an embodiment of the method, the interior of the vagina or cervix is rinsed with an appropriate solution prior to insertion of the device into the vagina, thereby staining the tissue in order to assist in the detection of pathologies.

All the above and other characteristics and advantages of the invention will be further understood through the following illustrative and non-limitative description of preferred embodiments thereof, with reference to the appended drawings.

### BRIEF DESCRIPTION OF THE DRAWINGS

FIG. 1 shows a schematic overall view of the device of the invention; and

FIG. 2 is a cross-sectional view showing schematically the main components of a basic embodiment of the device of the invention.

### DETAILED DESCRIPTION OF THE INVENTION

The invention is a system for performing a routine vaginal examination and a method comprising performing the examination and analyzing and interpreting the results. The examination is carried out using a medical imaging device that is optimally shaped to match the shape of the vaginal canal and comprises an imaging sensor, wide field of view optics, illumination means, electronic circuitry, and communication means. In addition a medical image and analysis (MIUA) unit comprising a processor and a dedicated MIUA algorithm is provided, either as part of the device or as a separate unit. The device and MIUA unit enable routine examination of the vagina to be carried out at any location by untrained persons, preferably by the woman herself in the privacy of her own home. The images acquired by the camera are transmitted to the MIUA unit by the communication means to determine whether “Eligible Views” of the examined area had been obtained and to provide an indication in accordance. After it has been determined by the MIUA unit that a predetermined set of “Eligible Views” has been obtained they are transmitted to an authorized center where trained medical personnel view, analyze, and interpret them.

The term “Eligible Views” as used herein means that a sufficient number of images having sufficient image quality and that include the desired region of interest (ROI) of the area being examined have been acquired in order to enable trained medical personnel to analyze and interpret them and make the required diagnosis. The ROI may be that of the cervix area, and may include also the vaginal canal.

The MIUA algorithm enables inexperienced users (in a preferred case the woman carrying out the self-examination) to acquire a complete set of “Eligible Views” that will be needed by the medical professional. After the device is turned on and inserted into the vagina, the algorithm compares the acquired video images to a predefined standard of image quality, if the acquired images do not meet the requirements of the predefined standard the algorithm instructs the processor and circuitry of the device to perform intelligent operations such as; adjustment of the illumination intensity or the exposure period of the imaging device. Such adjustments can be carried out automatically or manually by the user following instructions issued by the processor. The algorithm also checks the acquired images for the presence of the ROI to determine if a complete set of “Eligible Views” has been obtained. In the event that all of the views are not obtained, the algorithm provides guidance to the user, instructing her to adjust the device's angle, move the device in/out, right/left, etc. in order to focus on the desired area. As a consequence of the use of capabilities provided automatically by the MIUA algorithm, the woman operating the device is able to use it to acquire a complete set of high quality images without actually viewing the acquired video.

The invention transforms routine vaginal examinations from the realm of “See & Treat” procedures to that of “Tele-Medicine” or “Tele-Care” Services. Women may perform the inspection themselves at a location distant from their physician, save time, and avoid an unpleasant examination on the gynecologist chair and then send the “Eligible Views” for diagnosis using a network such as the internet.

For a better understanding of the invention and to show how it may be carried into effect, reference will now be made, purely by way of example, to the accompanying drawings. With specific reference to the drawings in detail, it is stressed that the particulars shown are by way of example and for purposes of illustrative discussion of embodiments of the present invention only, and are presented for the purpose of providing what is believed to be the most useful and readily understood description of the principles and conceptual aspects of the invention. In this regard, no attempt is made to show structural details of the invention in more detail than is necessary for a fundamental understanding of the invention. From the description taken together with the drawings it will be apparent to those skilled in the art how the several forms of the invention may be embodied in practice.

The main components of the device of the invention are:
a. an imaging sensor;
b. wide Field Of View optics;
c. illumination means;
d. communication means;
e. a processor;
f. a MIUA algorithm;
g. memory means;
h. an energy pack;
i. visual and/or audio display means;
j. operation buttons/switches;
k. an optional sterile disposable cover; and
l. one or more optional sensors.

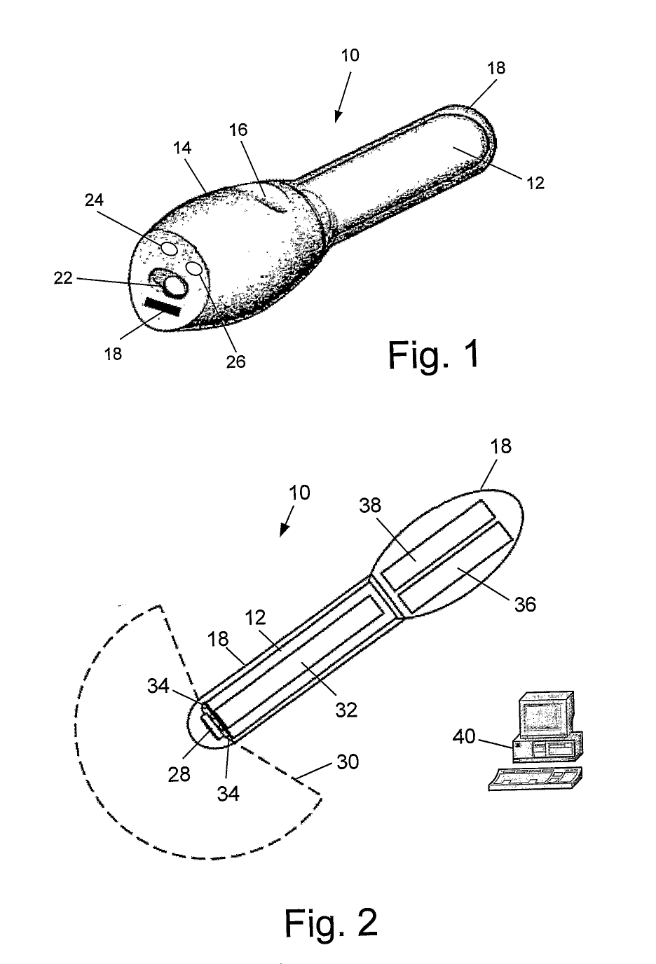

FIG. 1 shows a schematic overall view of the device 10 of the invention. Device 10 is comprised of an elongated insertion section 12, which is shaped so that it can be easily and smoothly inserted into the vagina, and handle 14 to assist the user to insert the insertion section into the vagina and to maneuver it in order to obtain “Eligible Views”. The handle is shaped and parts of its surface 16 are recessed or textured to enable easy griping of the device. Shown on the handle are communications port 20, on/off switch 22, a switch 24 that allows the user to transmit the “Eligible Views” for analysis, and a port 26 for direct connection of electrical energy from an external power source or for recharging an internal rechargeable battery pack.

The device is made of smooth material that is impervious to body fluids and is easily cleaned with soap and water and sterilized using alcohol or another suitable agent. The device can optionally be provided with a disposable sterile cover 18, which slides over at least the part of device 10 that is inserted into the vagina or comes in contact with the body of the person being examined. Although the invention is described herein in terms of self-use by a single individual, it is obvious that it can also be used by health care providers on many women in a clinical setting or can be shared by several women. In these situations the use of a disposable cover greatly simplifies the cleaning and sterilization process, reducing the risk of the spread of disease.

In some embodiments, one or more sensors or indicators may be attached to the outer surface of the insertion section 12 or the disposable cover 18 of the device in order to detect abnormalities in the secretions inside the vagina. An example of a physical parameter that can be measured is the level of Ph in the vagina, biosensors can be used to detect infections, and fibronectin level can be detected to evaluate the chances of premature delivery by a pregnant woman.

FIG. 2 is a cross-sectional view showing schematically the main components of a basic embodiment of the device of the invention. At the distal end of insertion section 12 is located a wide field of view (WFOV) lens system 28. The exact design of the WFOV optics is not a part of the present invention and lens system 28 can comprise a single optical element or be a combination of several elements. It is however required that the optical system 28 have a WFOV to assist the orientation and/or to increase the chance of detection of abnormalities by providing an overall view of the entire interior surface of the vaginal canal. Examples of different types of WFOV optical systems have been given by reference in the background section of this application and persons skilled in the art will have no trouble designing a suitable lens system.

Surrounding optical system 28 on the distal end of the device are illumination means 34, which in this case are several Light Emitting Diodes. The number and type of LEDs are selected to provide illumination of the entire wide Field Of View 30 provided by optical system 28.

Behind lens system 28 and illumination means 34 are located an electronic video camera and its associated electrical circuit, represented collectively by numeral 32. In preferred embodiments the imaging sensor of the camera, WFOV optical system, and the illumination means enable images to be gathered at different wavelengths in order to assist in the detection of pathologies. For example a black & white imaging based on a CCD or a CMOS chip sensor can be used. By illuminating the viewed area at different wavelengths a multi-spectral image can be produced which assists detection of abnormalities. In a similar manner, mechanical filters may be used in the device. As an example a blue filter assists in the detection of blood vessels. The filter functions may preferably be produced by software. Alternatively disposable covers can be supplied with different filters at the distal end, making it easy for the user to collect some images with one filter and then withdraw the device, change covers and reinsert the device to collect more images. Yet another option to improve detection of abnormalities is to rinse the interior of the vagina or the cervix with an appropriate solution to stain the tissue in order to assist in the detection of pathologies. At least the distal part of the insertion section 12 and cover 18 (if one is used) must be optically transparent to the wavelengths produced by illumination means 34 and to which the sensor of camera 32 are sensitive.

Shown in the handle 18 are communication means 36 for communicating with the MIUA unit 40 and energy pack 38 comprising, e.g. rechargeable batteries.

In preferred embodiments the invention enables use of the relatively simple device shown in FIG. 2 by basing the MIUA unit on a standard personal computer, which is provided with the MIUA algorithm that enables it to perform the image processing, the image understanding and the transmission of the “Eligible Views” for diagnosis, using standard networks. The MIUA algorithm can be installed on the computer by conventional means, e.g. from a CD or by download of files from the internet.

The communication means 36 on device 10 enables transmission of the acquired information to the computer 40 via wire communication, e.g. USB2 and FIRE WIRE 2 or wireless communication, e.g. BLUETOOTH and WIFI.

The MIUA algorithm runs on the acquired information to determine whether “Eligible Views” of the examined area have been acquired and to provide an indication to the user in accordance with the result of the determination.

In this embodiment the computer enables the initial processing of the acquired information by the MIUA algorithm in order to verify that “Eligible Views” had been acquired. In order to obtain a complete set of “Eligible Views”, the MIUA unit (the home computer) includes a designated algorithm which enables comparison between the information so far acquired in the images collected from the user's body and information that has been previously stored in the memory of the computer. Based on this comparison, the software uses the display of the home computer or sends voice commands to instruct the user how to maneuver device 10 in order to obtain the images needed to complete the “Eligible Views”. The software can include the capability to scan the entire interior of the vagina or to recognize and “zero-in” on specific areas, e.g. the cervix area and provide “Eligible Views” of that area only.

In addition to problems associated with aiming the camera, which can be corrected by the user, if she is properly instructed, other factors related to the illumination and optical parameters of the system may prevent the collection of a complete set of “Eligible Views”. Therefore, the MIUA algorithm also checks the quality of the images against predefined standards and enables modification of the definition of the parameters of electrical components of the circuitry within the device (not shown in the figures) in order to improve the image quality of the acquired information; e.g. the algorithm may enable automatic modification of the illumination intensity, the exposure time of the imaging sensor, the focus area, or provide optical or digital zoom.

Once the MIUA unit determines that the “Eligible Views” have been obtained, they can be transmitted, e.g. via the internet, to an authorized center for diagnosis.

In another embodiment the device may include an integral processor, comprising the MIUA algorithm; a memory chip; and integral means for communicating with the authorized center thus providing a completely self contained device not relying on a standard computer. In this embodiment it is preferable that there also be small display screen and speaker integrated into or attached to the handle to enable instructions to be sent to the user by the processor.

There are a variety of methods of providing a communication link between the device and the remotely located authorized center. For example, the communication might be channeled directly from the device to the authorized center or indirectly via the user's computer or via a docking station connected to the device. The communication can be wired or wireless; via a standard network, e.g. internet, cellular, or satellite; the communication might be analog or digital, compressed, selective or raw data. Privacy can be assured by assigning a unique ID address to each device. In a preferred embodiment, the “Eligible Views” are not forwarded automatically from the MIUA unit to the authorized center but the user determines if they should be sent or not. For the sake of brevity and since these and other methods are well known to those skilled in the art, no further elaboration of the communication methods shall be given herein.

The authorized center that receives the “Eligible Views” and other data might be the woman's physician or a specialized or general practice clinic that employs personnel that are trained to gather, document, classify, and diagnose the received information.

At the authorized center the acquired information may be studied by medical professionals that diagnose the “Eligible Views” by themselves. In preferred embodiments of the invention MIUA software is installed in the computers at the authorized center in order to allow some of the initial routine screening of the images to be preformed automatically. The MIUA software may also enable classification of the acquired information in such a manner that if a slight chance of an abnormality is detected, the entire set of “Eligible Views” is sent to a human specialist for diagnosis. The software at the authorized center is designed to provide convenient, user friendly display of the acquired information. In particular the wide field of view images acquired by the device during the examination assists the orientation within the image and may increase the chance for detection of abnormalities. In preferred embodiments the software allows different formats for documentation of the information, comparison to the results of previous examinations of the same woman, and also consolidation of data for wider statistical studies. The user interface at the authorized center allows at least some of the functions described to be performed automatically at the discretion of the operator.

In a preferred embodiment, the MIUA unit of the system enables automatic detection of predefined phenomenon. For example, after obtaining “Eligible Views” the software also enables analysis of the “Eligible Views” in order to automatically detect predefined abnormalities e.g. the presence of polyps. If no such abnormalities are detected the woman conducting the exam is informed and can decide that there is no need to send the “Eligible Views” to an authorized center for diagnosis. As another example, the software may be configured to provide a measurement of the opening of the cervix thereby providing a pregnant woman with an indication of when the birth of her baby is imminent.

The system and method of the invention have been conceived by the inventors such that, although they can be used by medical professionals such as doctors or nurses, they are simple enough to be carried out by inexperienced persons, ideally the woman herself. The MIUA algorithm and device are designed to provide step-by-step instructions whether aurally or visually during the course of the examination. What little instruction is needed to give the woman an overall view of the procedure and the confidence to perform the examination herself can be provided in a variety of ways; e.g. by means of written instructions supplied with the device, by receiving instructions from a trained health care person, or by watching an instruction video that can be supplied on a CD or downloaded from the internet.

The method of carrying out the examination is extremely simple. After activating the device by means of the on/off switch the user inserts the insertion section into her vagina. The camera automatically acquires images and transmits them to the image processing and analysis unit, either internal or a home computer. The MIUA algorithm analyzes the acquired images and adjusts parameters of the illumination means or the camera and provides directions to the user to adjust the position of the device in order to obtain “Eligible Views”. The MIUA unit instructs the user to turn off the device and to remove it from her vagina after it has determined that a complete set of “Eligible Views” has been obtained. The acquired “Eligible Views” have been saved in the memory of the MIUA unit and the user can now transmit them via the internet or some other means to her authorized center for diagnosis.

Although embodiments of the invention have been described by way of illustration, it will be understood that the invention may be carried out with many variations, modifications, and adaptations, without exceeding the scope of the claims.

1. A system for performing a routine vaginal examination comprising:
a medical imaging device comprising: an insertion section designed for insertion into the vagina, optics and an imaging sensor having a Wide Field of View (WFOV) capability; circuitry; illumination means; and communication means;
a medical image understanding and analysis (MIUA) unit comprising a processor, and a dedicated MIUA algorithm; and
an authorized center comprising image viewing software and human experts to analyze the images acquired by said device;
characterized in that said dedicated MIUA algorithm of said MIUA unit:
compares said acquired images to other cervical images to verify that sufficient images of the cervix of a woman being examined have been obtained;
compares said acquired images to a predefined standard of quality and, if necessary, automatically modifies the definition of parameters of the electrical components of said circuitry to improve the image quality;
saves the acquired images that meet said predefined standard and include all of said cervix of said woman being examined; and
provides an indication to the user carrying out said examination when a complete set of “Eligible Views” of said cervix of said woman being examined have been obtained.

2. A system according to claim 1, wherein the dedicated MIUA algorithm is adapted such that the routine vaginal examination can be carried out by an inexperienced user.

3. A system according to claim 2, wherein the inexperienced user is the woman being examined.

4. A system according to claim 1, wherein the device comprises an elongated insertion section shaped so that it can be easily and smoothly inserted into the vagina attached to a handle shaped to assist the user to insert said insertion section into the vagina and to maneuver it in order to obtain “Eligible Views”.

5. A system according to claim 1, wherein at least the distal part of the insertion section is optically transparent to the wavelengths produced by the illumination means and to which the imaging sensor is sensitive.

6. A system according to claim 1, wherein the device comprises a disposable sterile cover, which slides over at least the part of the device that is inserted into the vagina or comes in contact with the body of the person being examined.

7. A system according to claim 6, wherein at least the distal part of the disposable cover is optically transparent to the wavelengths produced by the illumination means and to which the imaging sensor is sensitive.

8. A system according to claim 1, wherein one or more sensors or indicators are attached to the outer surface of the insertion section of the device in order to detect abnormalities in the secretions inside the vagina.

9. A system according to claim 1, wherein the imaging sensor, the optics and the illumination means are adapted to gather images at different wavelengths to enhance detection or recognition of pathologies.

10. A system according to claim 1, wherein the imaging sensor is a black and white sensor and the illumination means provide illumination at different wave lengths to provide multi-spectral imaging to enhance detection or recognition of pathologies.

11. A system according to claim 1, comprising at least one filter to assist in detection of pathologies using the acquired information.

12. A system according to claim 11, wherein the filter function is provided by designated software.

13. A system according to claim 1, wherein the MIUA unit comprises a display and interface with the user.

14. A system according to either claim 1, wherein the MIUA unit is based on a standard personal computer on which is installed the MIUA algorithm.

15. A system according to claim 1, wherein the MIUA unit is an integral part of the medical imaging device.

16. A system according to claim 1, wherein the MIUA unit provides the user with instructions how to maneuver the device in order to obtain a complete set of “Eligible Views”.

17. (canceled)

18. A system according to claim 1, wherein the parameters are related to one or more of the following: illumination intensity, the exposure time of the imaging sensor, the focus area, optical zoom, or digital zoom.

19. A system according to claim 1, wherein, after a complete set of “Eligible Views” has been collected, the MIUA unit signals the user that the examination has been completed and the user can manually transmit data comprising at least said set to the authorized center.

20. A system according to claim 1, wherein, after a complete set of “Eligible Views” has been collected, the MIUA unit automatically transmits data comprising at least said set to the authorized center and signals the user that the examination has been completed.

21. A system according to of claim 19, wherein the data transmission, from the imaging device to the MIUA unit and from the MIUA unit to the authorized center, is performed over a standard communication network.

22. A system according to claim 19, wherein the device includes a unique ID address, which is transmitted with the data to the authorized center.

23. A system according to claim 1, wherein the MIUA unit is adapted to provide images of the entire interior of the vagina

24. A system according to claim 1, wherein the MIUA unit is adapted to recognize and provide images of specific areas in the vagina.

25. (canceled)

26. A system according to claim 1, wherein the MIUA unit enables measurement of the opening of the cervix before childbirth.

27. A system according to claim 1, wherein the human expert at the authorized center is the physician of the woman who was examined.

28. A system according to claim 1, wherein the authorized center comprises one or more computers containing software adapted to provide different formats for observation of the data received, for documentation of said data, for comparison of said data to the results of previous examinations of the same woman, and for consolidation of data for wider statistical studies by human experts.

29. A system according to claim 28, wherein the software is adapted to allow the computer to perform automatically at least some of the functions of the human experts.

30. A method for using the system according to claim 1 for performing a routine vaginal examination comprising:
the user turning on the medical imaging device;
said user inserting the insertion section of said medical imaging device into the vagina to be examined;
said user withdrawing said insertion section from said vagina upon receiving a signal from said MIUA unit that a set of “Eligible Views” has been obtained;
transmitting data comprising at least said set of “Eligible Views” to the authorized center;
said user turning off said medical imaging device; and
analyzing said transmitted data at said authorized center.

31. A method according to claim 30, wherein the user follows instructions from the MIUA unit to maneuver the insertion section to change the orientation of the imaging sensor within the vagina.

32. A method according to claim 30, wherein the user is the woman being examined.

33. A method according to claim 30, wherein the interior of the vagina or cervix is rinsed with an appropriate solution prior to insertion of the device into the vagina, thereby staining the tissue in order to assist in the detection of pathologies.

34. A system according to claim 6, wherein one or more sensors or indicators are attached to the outer surface of the insertion section of the device in order to detect abnormalities in the secretions inside the vagina.

35. A system according to claim 13, wherein the MIUA unit is based on a standard personal computer on which is installed the MIUA algorithm.

36. A system according to claim 13, wherein the MIUA unit is an integral part of the medical imaging device.

37. A system according to claim 20, wherein the data transmission, from the imaging device to the MIUA unit and from the MIUA unit to the authorized center, is performed over a standard communication network.

38. A system according to claim 20, wherein the device includes a unique ID address, which is transmitted with the data to the authorized center.
